(19) United States
(12) Patent Application Publication
(10) Pub. No.: US 2004/0224030 A1
(43) Pub. Date: Nov. 11, 2004

(54) Microsphere delivery systems

(76) Inventors: Venkatram R. Shastri, Lower Gwynedd, PA (US); Chien-Chun Yang, Shinchu (TW); Robert S. Langer, Newton, MA (US)

Correspondence Address: Choate, Hall & Stewart, Exchange Place, 53 State Street, Boston, MA, 02109, US

(21) Appl. No.: 10/430,817

(22) Filed: May 6, 2003

(51) Int. Cl. A61K 9/16; A61K 9/50

(52) U.S. Cl. 424/490

(57) ABSTRACT

Microspheres are provided that have controlled release profiles. The microspheres of the present invention include blended PLGA copolymers and a biologically active agent. Delivery of a biologically active agent to a specific in vivo location can be accomplished by administration of the microspheres in a pharmaceutical composition. Microspheres of calcitonin are particularly provided for the treatment and prevention of osteoporosis or the augmentation of hormone replacement therapy.

### BACKGROUND OF THE INVENTION

The development of methods and devices for the encapsulation of biologically active agents has been a challenging area of scientific research over the last decade. A variety of encapsulation methods by which compounds can be encapsulated to increase the circulating half-life are available in the form of microparticles or microspheres. Biologically active and pharmaceutically active agents are typically encapsulated within a biocompatible, biodegradable, wall-forming material such as a polymer to provide sustained or delayed release of drugs or other active agents. The material to be encapsulated is generally dissolved, dispersed, or emulsified in a solvent containing the wall forming material. Once the material (drug or other biologically active agent) is encapsulated, the solvent is removed from the microparticles.

An important factor in the successful treatment of long-term chronic disease, such as osteoporosis, diabetes, asthma, hepatitis, and arteriosclerosis etc., is patient compliance to the prescribed treatment regimen. However, the protein and peptide drugs often used to treat chronic diseases typically require multiple doses by injection, which are painful to the patient and often dramatically decreases compliance. Although a variety of delayed release microspheres of poly(DL-lactide-co-glycolide) (PLGA) copolymers are available in the art, there exists the need for improved PLGA microspheres containing biologically active agents that have controlled release profiles.

### SUMMARY OF THE INVENTION

The present invention provides blends of biodegradable polymers containing biologically active agents. Preferably, the composition of the blend modulates the release kinetics of the microspheres. More particularly, the present invention provides microspheres containing biologically active agents that have controlled release profiles, preferably delayed release profiles, which provide prolonged release of a biologically active agent.

According to the present invention, the release kinetics modulated include the rate of release of the biologically active agent from the microsphere; the cumulative release of the biologically active agent from the microsphere; the duration of release of the biologically active agent from the microsphere; the burst effect of the biologically active agent from the microsphere, i.e., the timing, amount, or duration of a first or second burst or pulse.

The compositions of the invention include microspheres having blends of polyhydroxy acids and copolymers thereof and biologically active agents. Some preferred polyhydroxy acids include polylactic acid and polyglycolic acid, and copolymers thereof. Certain preferred copolymers include poly(DL-lactide-co-glycolide) (PLGA) copolymers or mixtures of PLGA copolymers and biologically active agents. In other preferred embodiments, the blends of poly-ε-lactone are combined with biologically active agents. Any biologically active agent may be used in the present invention. Some preferred biologically active agents include calcitonin, estrogen, progesterone, and combinations of estrogen and progesterone. The PLGA copolymers used to make the microspheres may further include end groups such as hydrophilic acid end groups or lipophilic ester end groups.

Some exemplary microspheres include microspheres of blended copolymers in combination with a biologically active agent. Without limitation, some particularly preferred microspheres combine a biologically active agent with a blend of PLGA copolymers including 25-80% poly(D,L-lactide-co-glycolide) having a 50%:50% ratio of racemic lactide DL to glycolide and a molecular weight of 20 Kdal (“RG502”), and 20-75% poly(D,L-lactide-co-glycolide) having a 75%:25% ratio of racemic lactide DL to glycolide and a molecular weight of 20 Kdal (“RG(75/25)”. Other preferred microspheres combine the biologically active agent with a blend of copolymers such as RG(75/25) and RG502 in ratios of 1:4, 1:1, or 3:1.

The present invention further provides pharmaceutical compositions containing the inventive microspheres. Any agent having therapeutic biological activity may be included in the microspheres. For example, certain of the pharmaceutical compositions are useful for the treatment of osteoporosis, such as those that include calcitonin as the biologically active agent. Other pharmaceutical compositions can be used for hormone replacement therapy. For example, microspheres that contain estrogen, progesterone, or a combination of estrogen and progesterone can be formed into pharmaceutical compositions for the treatment or prevention of menopausal symptoms in post-menopausal women.

In other preferred embodiments, the invention provides methods of generating controlled release microspheres by blending PLGA copolymers of varying molecular weight or varying lactide to glycolide ratios. In certain preferred embodiments, the molecular weight of the copolymers varies generally from 20 to 100,000 KDal. The ratio of lactide to glycolide may varies from 1:1, 1:2, 1:3, 1:4, to 1:5.

Related embodiments provide methods for controlling the rate, amount, duration, or timing of release of a biologically active agent from the microspheres by varying the type of PLGA copolymer or copolymers in the microspheres. In one preferred embodiment, the release profile is controlled by adjusting the ratio of a first PLGA copolymer to a second PLGA copolymer. In yet other preferred embodiments the burst effect of the microspheres is controlled by varying the type or blend of PLGA copolymers in the microspheres.

In a final embodiment, the present invention provides methods of making microspheres of about 20 to 30 micrometers by a) contacting a solution of biologically active agent with a solution of PLGA copolymer to form a biologically active agent:copolymer mixture, wherein the solution of PLGA copolymer includes a blend of copolymers 25-80% RG502 and 20-75% RG(75/25); b) emulsifying the mixture by sonication to generate a first emulsified solution; c) emulsifying the first emulsified solution by homogenization to form a double emulsified solution; and d) separating the microspheres from the double emulsified solution. For example, some preferred biologically active agents include calcitonin, estrogen, progesterone, and combinations of estrogen and progesterone.

### Definitions

By “microparticles” or “microspheres” is meant solid particles that contain an active agent dispersed or dissolved within a polymer that serves as the matrix of the particle. The polymer is preferably biodegradable and biocompatible. By “biodegradable” is meant that a material should degrade by bodily processes to products readily disposable by the body and should not accumulate in the body. By “biocompatible” is meant not toxic to the body. A pharmaceutical that is biocompatible is not carcinogenic and does not significantly induce inflammation in body tissues.

By “blend” of polymers or copolymers or “blended” polymers or copolymers is meant that more than one type of polymer or copolymer is included in a mixture of polymers or copolymers. As but one example, a blend of copolymers, according to the invention, is a blend of RG502 and RG(75/25).

A “biologically active agent” is a compound that when added to an in vitro or in vivo biological system induces a biological response. By “biological response” is meant a physiological reaction that occurs in vivo or in vitro, preferably in vivo. A biological response is often measurable by an in vitro or in vivo assay. The bioactive agents of the invention are preferably capable of inducing a biological response. Examples of biologically active agents include proteins, peptides, proteins that are enzymes, genetic products (for example, polynucleotides, oligonucleotides and other nucleic acids such as locked nucleic acids (LNAs) and peptide nucleic acids (PNAs) see, e.g., Demidov, Trends Biotechnol. (2003) January; 21 (1):4-7) and natural or synthetic chemical compounds. Preferably the biologically active agent has beneficial therapeutic of pharmaceutical activity in vivo.

“By weight %” or “% by weight” is meant parts by weight per total weight of a microsphere. For example 10% by weight of a biologically active agent would mean 10 parts of biologically active agent by weight and 90 parts polymer.

### BRIEF DESCRIPTION OF THE DRAWING

FIG. 1 is a graph showing the cumulative amounts of calcitonin released from microspheres made of different PLGA copolymers.

FIG. 2 shows three graphs illustrating the release profiles of microspheres containing BSA as follows: a 1:1 ratio of RG502 to RG(75/25) (panel A), RG502 (panel B), and RG(75/25) (panel C).

FIG. 3 is a graph showing the cumulative amounts of BSA released from microspheres made of different blends of PLGA copolymer.

FIG. 4 shows two graphs illustrating the release profiles of RG503H (panel A) and RG503 (panel B), which have different end groups.

FIG. 5 shows four graphs of release amount vs. release time of BSA from microspheres of different PLGA copolymers: RG502 (panel A), RG(75/25) (panel B), RG503H (panel C), and RG503 (panel D).

FIG. 6 shows two graphs of release amount vs. release time of BSA from microspheres of blended PLGA copolymers: 75% RG(75/25) and 25% RG502 (panel A), and 50% RG(75/25) and 50% RG502. (All % are % by weight).

FIG. 7 is a graph showing the cumulative amounts of calcitonin released from microspheres made of different PLGA copolymers.

FIG. 8 is a graph showing the amount and rate of release of calcitonin from microspheres from PLGA copolymers and blends of PLGA copolymers.

FIG. 9 is a graph showing the release of calcitonin from microspheres of blended PLGA copolymers.

FIG. 10 is a graph showing the amount and rate of release of calcitonin from microspheres of PLGA copolymers and blends of PLGA copolymers.

FIG. 11 shows graphs that illustrate the release profiles of microspheres made of various PLGA copolymers (panels A-E).

### DETAILED DESCRIPTION OF THE INVENTION

The present invention provides microspheres having controlled release profiles. In one preferred embodiment, the present invention provides microspheres that contain a blend of biodegradable polymers and a biologically active agent, wherein the composition of the blend modulates the release kinetics of the microspheres. Modulated release kinetics result in controlled release profiles, wherein the kinetics of release of a biologically active agent from a microsphere that includes a specific blend of polymers has certain release profile. Those skilled in the art will appreciate that the invention relates to controlling or modulating certain characteristics of microspheres, such as the kinetics of release of a biologically active agent, by blending polymers together to generate copolymers with specific characteristics.

The present invention further provides methods of preparing microspheres having controlled release profiles. According to the present invention, the timing, rate, quantity, and/or duration of release of a biologically active agent from a microsphere can be controlled or modulated by optimization of the microsphere copolymer ratio. In certain preferred embodiments, the microspheres contain a blend of particular copolymers having different lactide to glycolide ratios. Without limitation, the lactide:glylcolide ratio determines the release profile of the microsphere.

In particularly preferred embodiments, the microspheres of the invention include protein or peptide biologically active agents, which are normally denatured in the gastric environment or degraded by biological enzymes in vivo. Microspheres of the invention that contain protein or peptide biologically active agents have release profiles that decrease or negate the need for multiple injections of the protein or peptide therapeutic into the patient. Preferably, the inventive microspheres can be implanted into a site in vivo by injection so that fewer doses of the protein or peptide are required. In a particularly preferred embodiment, only one dose is required. This advantage is likely to increase patient compliance to treatment regimens that typically require multiple doses of protein or peptide therapeutics.

Microsphere Compositions

The microspheres of the invention include blends of polymers and a biologically active agent. As but one example, the present invention provides PLGA copolymers and a biologically active agent. PLGA is a biocompatible, biodegradable, and clinically acceptable material. In preferred embodiments, the microspheres of the invention are blends of more than one, preferably two, PLGA copolymers and a biologically active agent. According to certain preferred embodiments, the blends of PLGA copolymers include PLGA copolymers that have different lactide to glycolide ratios. The blends of copolymers may include any PLGA copolymer.

For example, some non-limiting blends of copolymers include poly(D,L-lactide-co-glycolide) having a 50%:50% ratio of racemic lactide DL to glycolide and a molecular weight of 20 Kdal (“RG502”), poly(D,L-lactide-co-glycolide) having a 50%:50% ratio of racemic lactide DL to glycolide and a molecular weight of 30 Kdal (“RG503”), poly(D,L-lactide-co-glycolide) having a 50%:50% ratio of racemic lactide DL to glycolide and a molecular weight of 20 Kdal and having a hydrophilic acid end group (“RG503H”), and poly(D,L-lactide-co-glycolide) having a 75%:25% ratio of racemic lactide DL to glycolide and a molecular weight of 2 (“RG(75/25)”), which can be purchased commercially from Boehringer Ingelheim, or equivalents thereof.

Those skilled in the art will appreciate that other biodegradable polymers may be substituted for or mixed with the PLGA compositions described herein. Exemplary biodegradable polymers include collagen-GAG, collagen, fibrin, PLA, polyesters, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides. Mixtures, adducts, and co-polymers of the above may also be employed for use according to the invention.

According to the present invention, certain preferred microspheres have ratios of RG(75/25) to RG502 of 1:4, 1:1, or 3:1. The invention further provides blends of copolymers containing 25-80% RG502 and 20-75% RG(75/25). Those skilled in the art will appreciate that any percent weight of RG502 and RG(75/25) that fall within these parameters are within the scope of the present invention. For example, those skilled in the art will appreciate that the quantities of 25%, 35%, 45%, 55%, 65%, or 75% RG502 may be combined with the quantities 75%, 65%, 55%, 45%, 35%, or 25% RG(75/25), respectively. Those skilled in the art will further appreciate that the quantities of 20%, 30%, 40%, 50%, 60%, or 70% RG(75/25) may be combined with the quantities 80%, 70%, 60%, 50%, 40%, or 30% RG502, respectively.

As mentioned above, the microspheres of the present invention include microspheres having varying ratios of lactide to glycolide. As but one non-limiting example, microspheres may have a lactide to glycolide ratio of 1:3, 1:4, or 1:5. According to the present invention, the choice of a particular PLGA copolymer determines the release profile of a blended copolymer. In one particular embodiment, microspheres of PLGA copolymers release different cumulative amounts of the biologically active agent. According to the invention, microspheres having a higher ratio of glycolide than lactide release higher cumulative amounts.

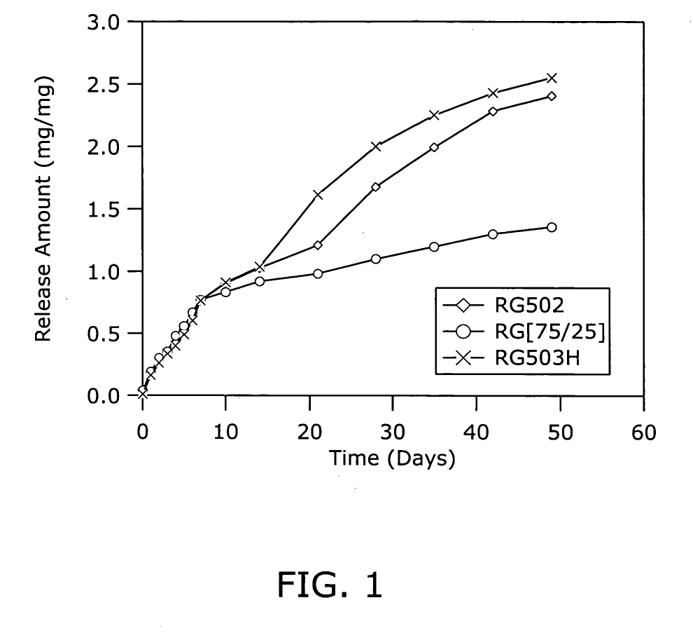

In related embodiments, the blended PLGA copolymers differ in the rate of release of a biologically active agent from the microsphere. In preferred embodiments, blends of PLGA copolymers display a slower rate of release than either of the original two materials individually. One particularly preferred blend that displays a reduced rate of release is a blend of RG502 with RG(75/25). The rate of release of such blended RG502:RG(75/25) copolymers is slower than the rate of release of either RG502 or RG(75/25) alone (see FIG. 1).

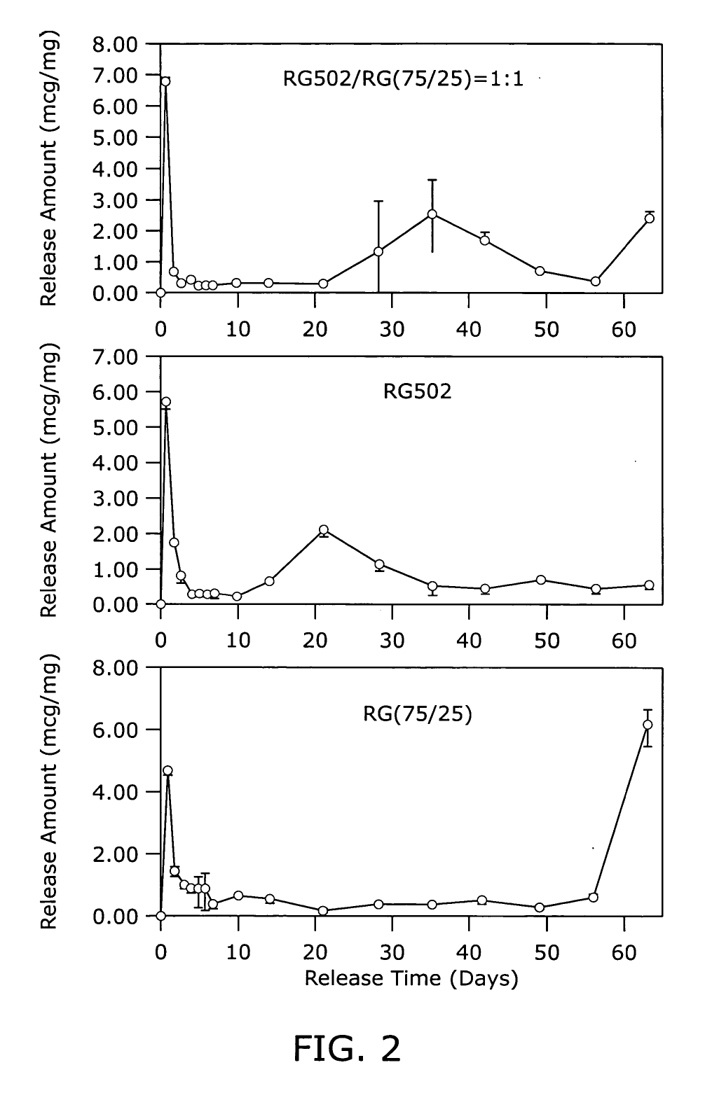

Other embodiments of the invention hinge on the discovery that manipulating the molecular weight of the microspheres controls the multi-phasic behavior of the microspheres. In particular, changing the molecular weight composition of the microspheres can alter the burst effect of the microspheres. More particularly, the quantity, duration, or timing of a first burst or second burst, also referred to herein as a first pulse or a second pulse, can be altered by the PLGA copolymer composition of the microsphere. Microspheres made with lower molecular weight PLGA (e.g., PLGA RG502, molecular weight 20 KDal) show higher first burst than microspheres made with higher molecular weight PLGA (e.g., 30 KDal). This trend can be extended to microspheres made with even higher molecule weight PLGA (e.g., 100,000 KDal). In addition, microspheres made with lower molecular weight PLGA have a delayed second burst. As shown in FIG. 2, the timing of the second pulse can be shifted over a two-week period by adjusting the ratio of copolymer used in the microsphere.

In related embodiments, the timing of the first or second burst can be shifted about 1 day, 2, days, 3 days, 4, days, 5, days, 6 days, 7 days, 8 days, 9 days, or 10 days, preferably about 10-15 days, more preferably, about 15-20 days, most preferably about 30 to 40 days. The timing of the first or second burst may also be shifted more than 40 days, e.g., 40-60 days. Those skilled in the art will appreciate that the timing of the first or second burst can be any number of days or half days within these parameters.

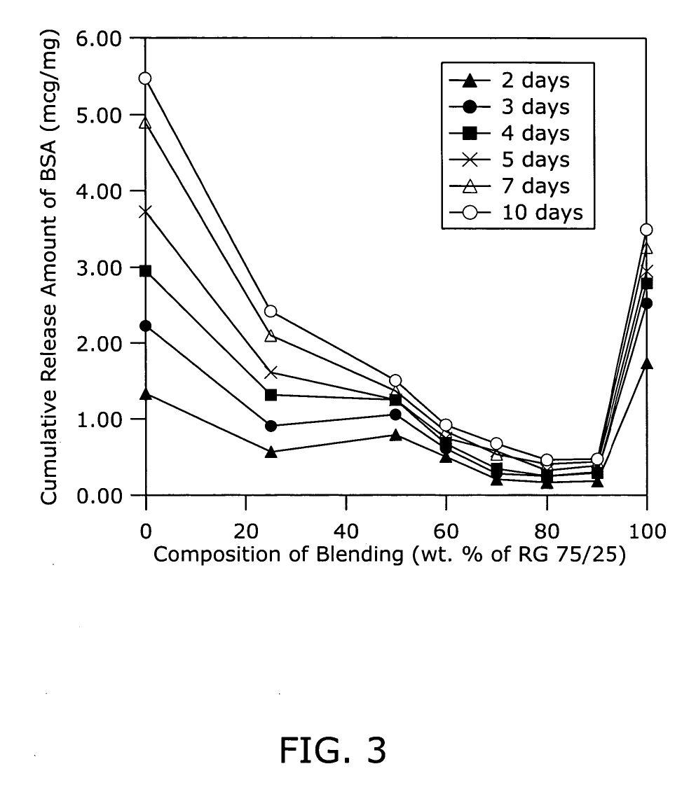

Finally, different blends of PLGA copolymers (e.g., RG(75/25) and RG502) can have shorter or longer durations of release depending of the particular composition of the blended PLGA copolymer. As shown in FIG. 2, the burst of blended PLGA copolymers may extend over longer periods of time than unblended PLGA copolymers. Sustained release from blended microspheres was attained for BSA for over 60 days. The results obtained for BSA were found to be applicable to calcitonin, showing that the BSA model can be applied to other protein therapeutics. FIG. 3 illustrates the release profiles of blended PLGA microspheres containing RG(75/25) to RG502.

The microspheres of the invention range in size from about 20 nanometers to 300 micrometers. In certain preferred embodiments, the microspheres of the invention fall within the range of 30 to 40 micrometers. The diameter of the microsphere is modulated depending on the desired alteration in release kinetics. The diameter of the microsphere may effect, for example, the cumulative amount of release, the rate of release, or the quantity, duration, or timing of a first burst or second burst. For example, the diameter may be altered in order to change the timing of the first and/or second burst peaks. The diameter may be further modulated depending on the mode of delivery or where the microsphere is going to be deposited in the body. For example, if the microsphere is going to be delivered via a catheter, it can be about 100 micrometers in diameter. Alternatively, if the microsphere is going to be deposited in the perivascular region, this region can only accommodate a microsphere having diameter of about 20 micrometers. Interstitial spaces, such as in the hippocampus, require particles as small as possible, e.g., within the 20 nanometer range. The diameter may further be modulated based on the modality of treatment. For example, if the treatment requires a quick release of the biologically active agent, the microsphere may be of a smaller diameter. If a slower release profile is required, a larger diameter may be required.

Some exemplary blends of copolymers that fall within either size range are compositions of microspheres that include blends of PLGA copolymers having a 1:4 ratio of RG(75/25) to RG502, a 1:1 ratio of RG(75/25) to RG502, or a 3:1 ratio of RG(75/25) to RG502, together with a biologically active agent, although, those skilled in the art will appreciate that these ratios can be modified to obtain desired release kinetics according to the invention. In certain preferred embodiments, the bioactive agent is calcitonin. Other preferred bioactive agents of the invention include estrogen, progesterone, or combinations of estrogen and progesterone.

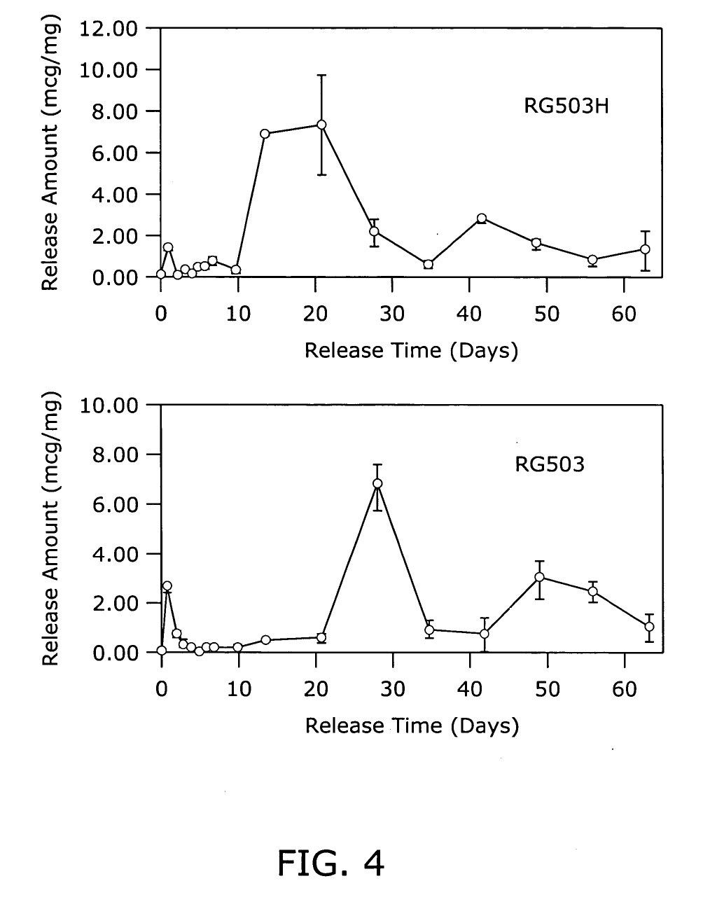

The microsphere compositions of the present invention may further include end groups such as hydrophilic acid end groups or lipophilic ester end groups. Some preferred PLGA copolymers have hydrophilic end groups. Other preferred PLGA copolymers have lipophilic ester end groups. For example, the RG503 system (Boehringer Ingelheim) provides 1:1 copolymers of di-lactic and glycolic acid with a molecular weight of 30 KDa that have different end groups. FIG. 4 shows that RG503H, which has a hydrophilic acid end group, has an earlier onset of a broad second pulse (between 10 and 20 days earlier) than RG503, which lacks an end group. In contrast, RG503, which has a lipophilic ester end group, has a sharper second pulse with a much later onset (between 20 and 30 days). Those skilled in the art will appreciate that any end group that alters the release profile of a microsphere can be attached to a copolymer used in the present invention. Those skilled in the art will further appreciate that any one of the PLGA copolymers that make up the inventive blended PLGA copolymers may also include an end group.

The hydroxyl and carboxylate end groups on PLGA and other polyesters provide sites to which molecules may be attached to modify the release properties of the material. For example, tert-butyl, benzyl, or other hydrophobic groups may be added to the polymer. Polar organic groups such as methoxy also facilitate adjustment of both the degradation rate and hydrophilicity. In contrast, addition of hydrophilic groups, for example, sugars, at these sites would increase the solubility of hydrophilic drugs within the polymer. Acids may also be added to the polymer to modify the properties of the polymer. For example, molecules with carboxylic or phosphoric acid groups or acidic sugars may be added. Charged groups such as sulfates and amines may also be attached to the polymer. For example, a charged amino acid such as arginine or histidine may be attached to the polymer to modify the release profile. Step growth polymers have reactive end groups that can be easily modified through standard organic chemistry reactions. Attachment of such non-protein organic or inorganic groups to the polymer modifies the hydrophilicity, the release profile, and the degradation rate of the polymer. Protecting group chemistry may also be used to modify the hydrophilicity of the material. One skilled in the art will recognize that a wide variety of non-protein organic and inorganic groups may be added to or substituted for the hydroxyl groups in the polymer to modify its properties. Exemplary functional groups are also described in March, Advanced Organic Chemistry. Fifth edition, John Wiley & Sons, Inc., New York, 1995, the entire contents of which are incorporated by reference herein.

Methods of Making Microspheres and Microparticles

The present invention provides methods of modulating or controlling the kinetics of release of a biologically active agent from a microsphere by blending polymers together to generate blended copolymers. For example, in one preferred embodiment, the present invention provides methods of modulating or controlling the kinetics of release of a biologically active agent from a microsphere by blending PLGA copolymers of varying molecular weight to form copolymers. In preferred embodiments, the microspheres are about 20 nanometers to about 300 micrometers. In certain preferred embodiments, the present invention provides methods of controlling the rate of release of a biologically active agent from a microsphere. In other preferred embodiments, the present invention provides methods of controlling the cumulative amount of a biologically active agent released from a microsphere. In yet other preferred embodiments, the present invention provides methods of controlling the duration of release of a biologically active agent from a microsphere. In each of these embodiments, the methods are preformed by varying the type and/or ratio of PLGA copolymer in the microsphere.

In another aspect, the present invention also provides methods of controlling the timing and/or rate of release of a biologically active agent from a microsphere by varying the ratio of a first and a second PLGA copolymer in the microsphere. The present invention further provides methods of controlling the cumulative amount of biologically active agent released from a microsphere by varying the ratio of a first and a second PLGA copolymer in the microsphere. In related embodiments, the present invention provides methods of controlling the duration of release of a biologically active agent from a microsphere by varying the ratio of a first and a second PLGA copolymer in the microsphere. In other related embodiments, the burst effect may also be controlled by varying the ratio of a first and a second PLGA copolymer in the microsphere, wherein the burst effect comprises a first pulse and a second pulse. According to the present invention, the ratio of the first and second PLGA copolymers in the microsphere may further control the timing, amount, or duration, of the first or second pulse.

According to certain preferred embodiments of the present invention the first and second PLGA copolymers are blended to form microspheres that have different lactide to glycolide ratios. Controlling the timing, amount, rate, duration, or burst effect of release of a biologically active agent from a microsphere may include varying the lactide to glycolide ratio of the first and/or second PLGA copolymer in the mixture. Some exemplary referred ratios of lactide to glycolide in a blended PLGA copolymer include ratios of 4:1, 3:1, 2:1, 1:1, 1:2, 1:3, 1:4, and 1:5. In particularly preferred embodiments, adjusting the ratio of lactide to glycolide controls the cumulative amount of biologically active agent released from the microsphere. Specifically, increasing the ratio of glycolide to lactide increases the cumulative amounts released from the microsphere. Lactide to glycolide ratios that result in increased release of the biologically active agent include, for example, 1:2, 1:3, 1:4, or 1:5.

The present invention further provides methods of controlling the burst effect of a microsphere by varying the type and/or ratio of PLGA copolymer in a microsphere. According to the present invention, the burst effect may include a first pulse and second pulse. By varying the type of PLGA copolymers in the microsphere, several parameters of the pulse can be manipulated, including the timing, amount, or duration of the pulse. The methods of the present invention may be used to control the timing, amount, or duration of either the first or the second pulse.

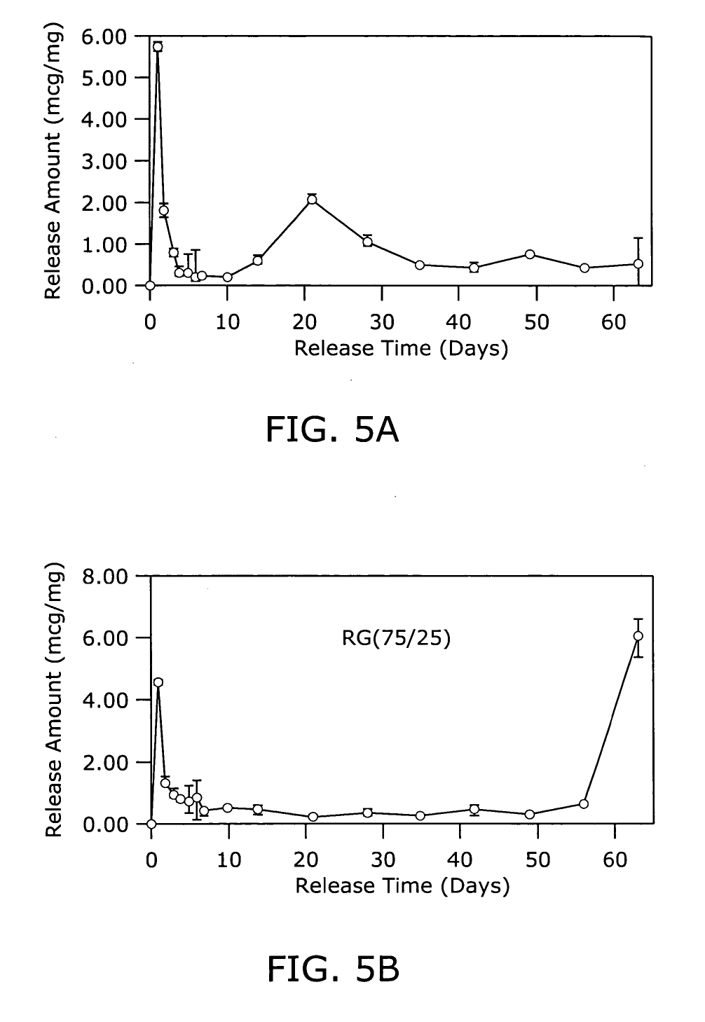

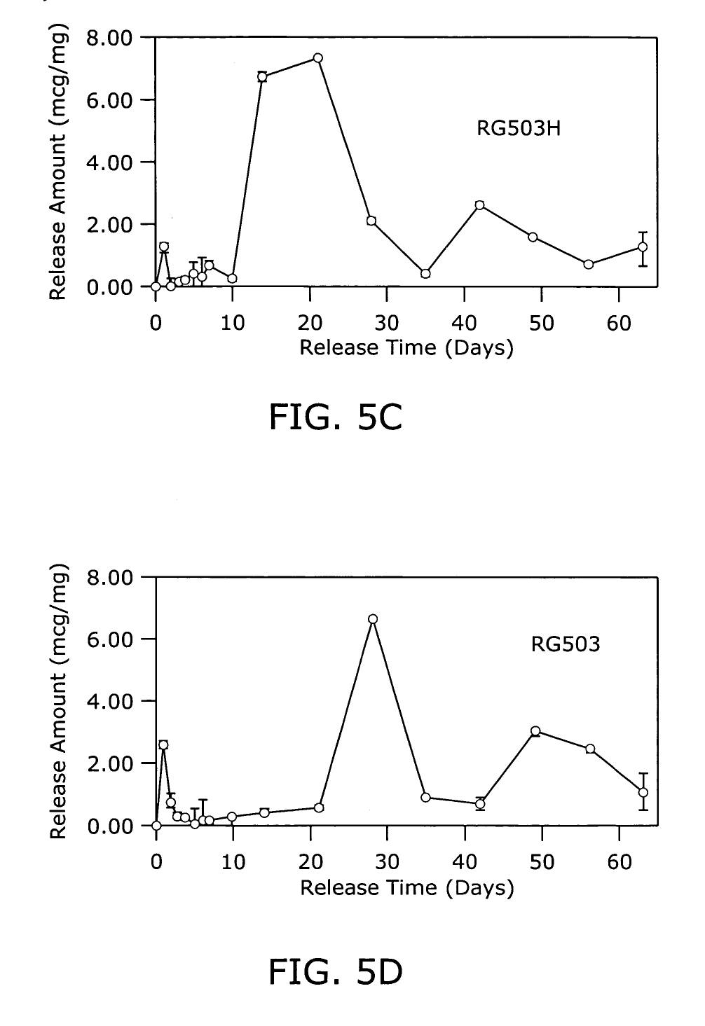

In related embodiments, the timing of a second pulse (or burst) is controlled by adjusting the ratio of PLGA copolymers in a blended PLGA copolymer. As demonstrated herein, the timing of the second pulse is delayed by preparing a blend of 75% RG(75/25) and 25% RG502 (3:1). A comparison of FIGS. 5 (panels A and B) and 6 (panel A), shows that a 3:1 ratio of RG(75/25) to RG502 shifts the second pulse from about 21 days to about 35 days, a shift of 14 days.

Generally, the methods of the invention involve blending together PLGA copolymers RG(75/25) and RG502. Exemplary ratios of RG502 to RG(75/25) include 1:4, 1:1, and 3:1 RG(75/25) to RG502. The invention further provides methods of forming blends of copolymers, wherein the resulting microsphere contains about 25-80% RG502 and about 20-75% RG(75/25). Without limiting the invention to these particular ratios, those skilled in the art will appreciate that any percent weight of RG502 and RG(75/25) that result in a particular desired release kinetics within the scope of the present invention. For example, those skilled in the art will appreciate that the quantities of 25%, 35%, 45%, 55%, 65%, or 75% RG502 may be combined with the quantities 75%, 65%, 55%, 45%, 35%, or 25% RG(75/25), respectively. Those skilled in the art will further appreciate that the quantities of 20%, 30%, 40%, 50%, 60%, or 70% RG(75/25)may be combined with the quantities 80%, 70%, 60%, 50%, 40%, or 30% RG502, respectively. Similar mixtures outside these ratio may be formulated based on the teachings of the present invention.

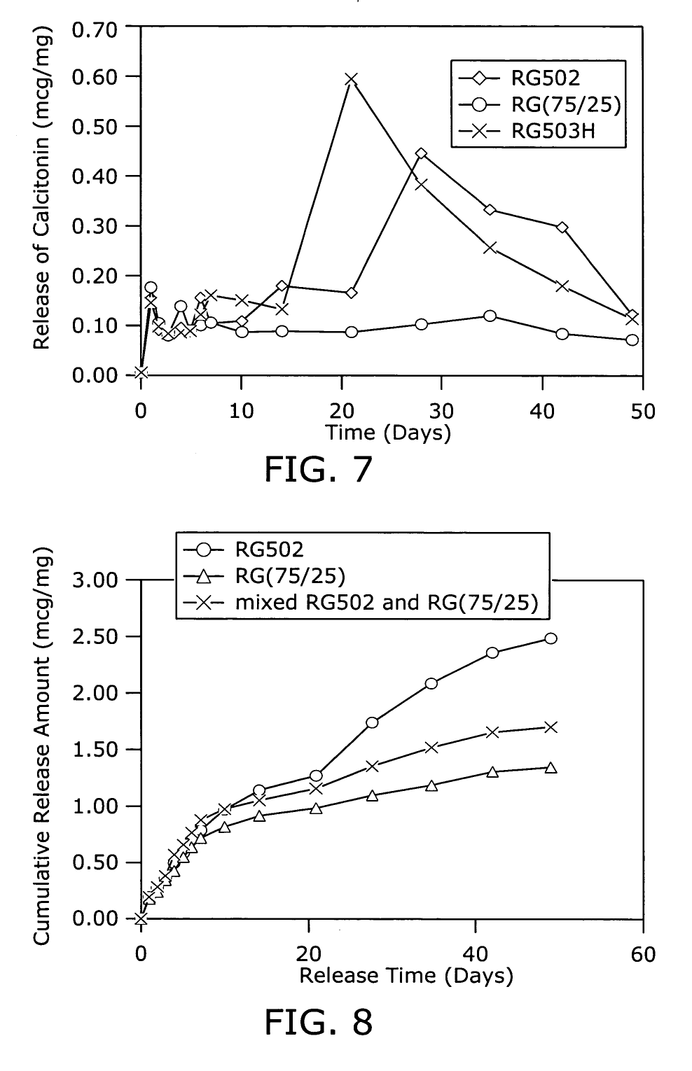

As described above, the type and/or ratio of PLGA copolymer that comprises the microspheres can control the cumulative amount of biologically active agent released from the microspheres. In preferred embodiments, different types of PLGA copolymers, which have different lactide to glycolide ratios, cause different amounts of biologically active agent, e.g., calcitonin, to be released from the microspheres. In one preferred embodiment, blending PLGA copolymers that have a higher ratio of glycolide than lactide increases the amount of biologically active agent released from the microsphere. For example, as shown in FIG. 1 and FIG. 7, the amounts of calcitonin released from RG502 and RG503H microspheres are higher than from RG(75/25) microspheres.

As described herein, the present invention further provides methods of controlling the duration of release of a biologically active agent from a microsphere by controlling the type and/or ratio of PLGA copolymer that comprises the microsphere. As but one example of the type of PLGA copollymer influencing the duration of release, the release period of RG(75/25) can continue for more than 7 weeks.

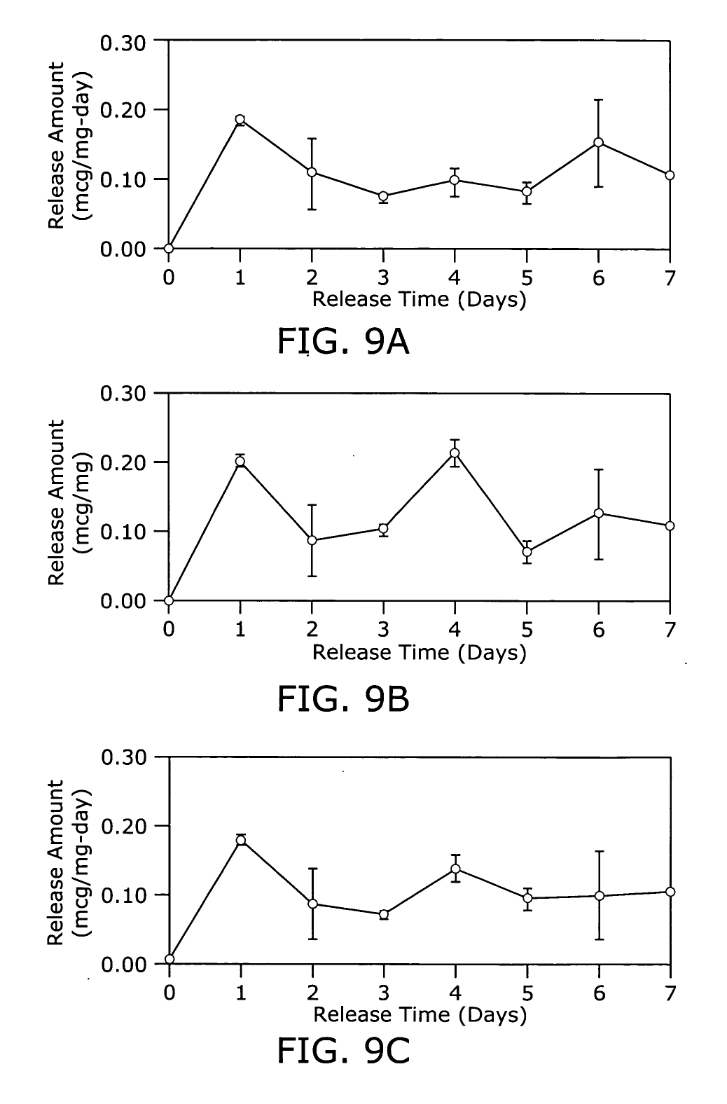

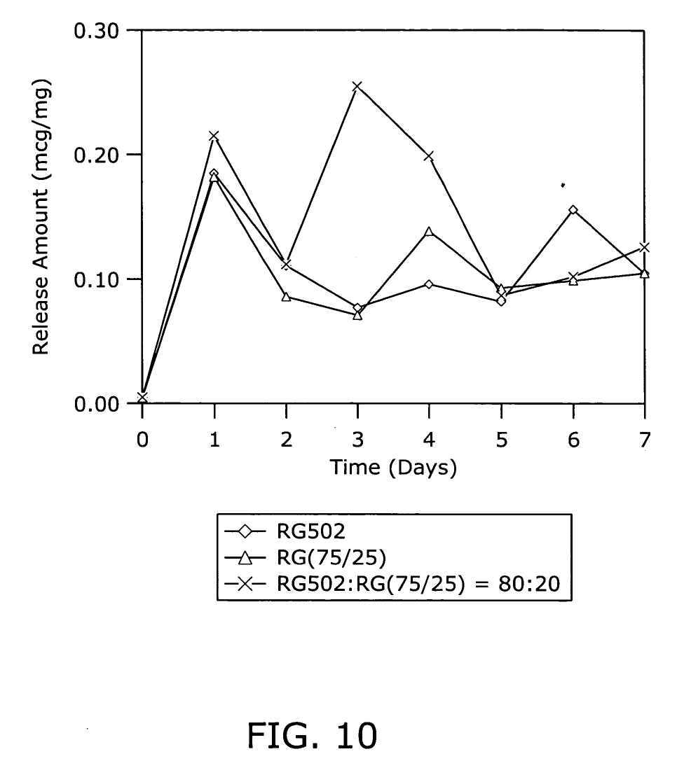

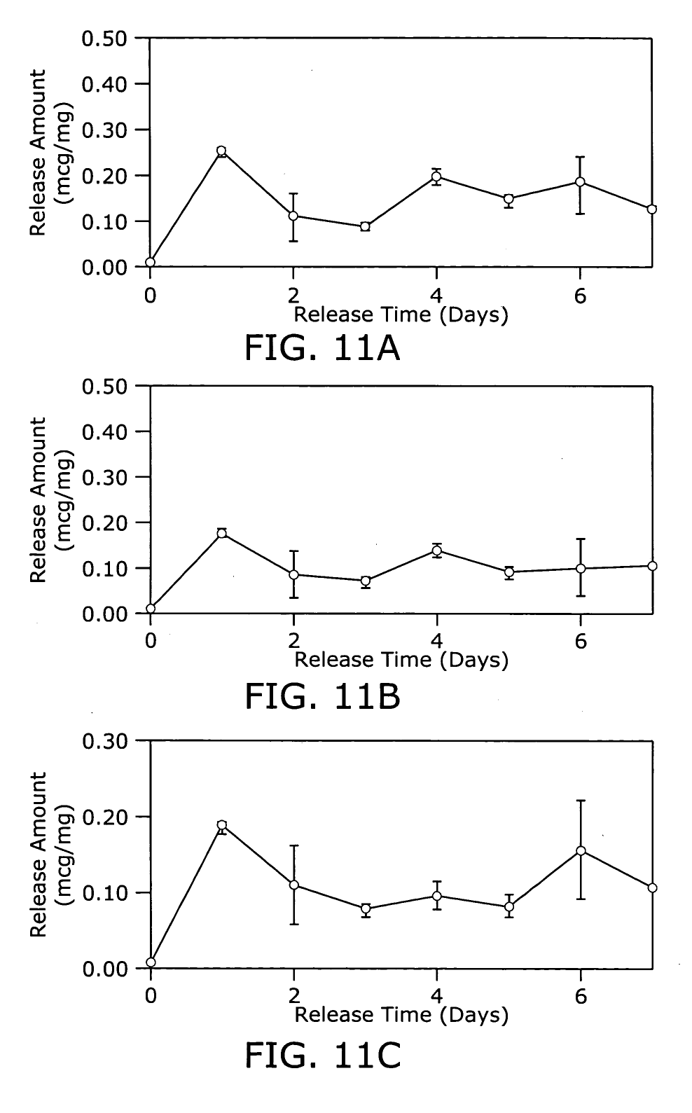

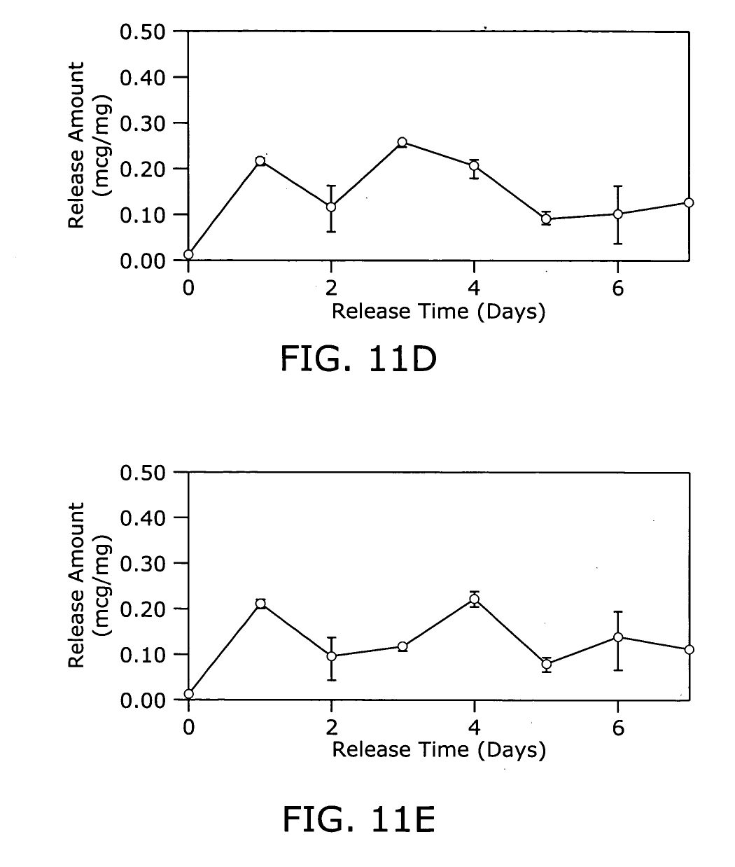

In other preferred embodiments, the invention provides methods of decreasing the amount of biologically active agent released from a first PLGA copolymer and decreasing the amount of biologically active agent released from a second PLGA copolymer by combining the first and second PLGA copolymers. For example, some mixtures of PLGA copolymers release an amount of biologically active agent that is intermediate between that of each of the PLGA copolymers individually (see FIG. 8 and FIG. 9). In addition, certain PLGA copolymers release higher amounts of biologically active agent (calcitonin) than the un-mixed PLGA copolymers (see FIG. 10).

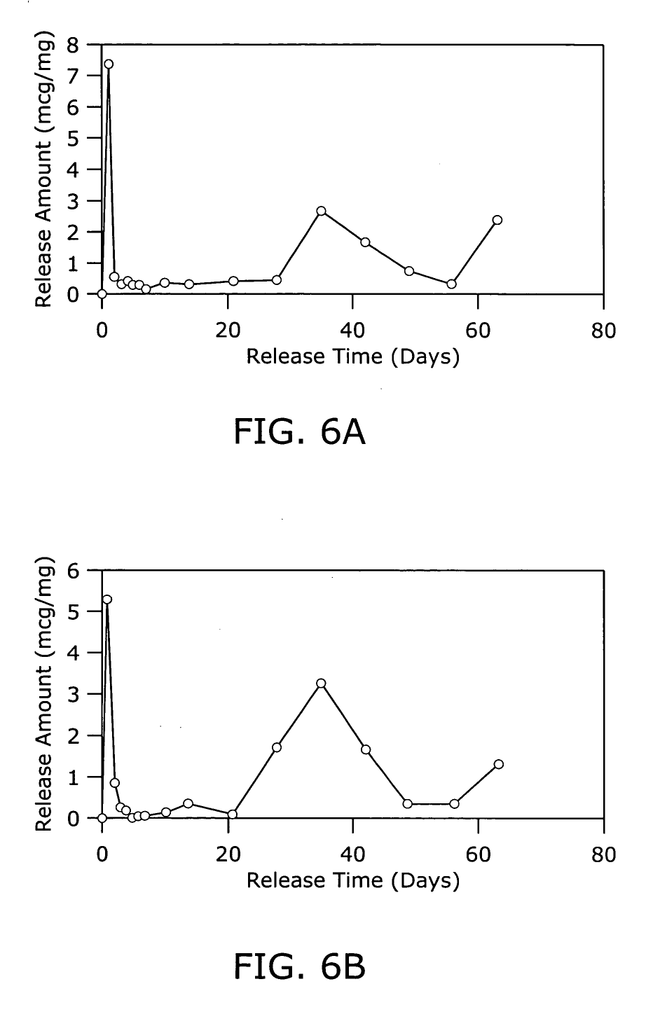

In related embodiments, the present invention provides methods for controlling the rate of release of a biologically active agent from a PLGA copolymer that include varying the type and/or ratio of PLGA copolymer selected for making the microsphere. For example, the rate of release of a biologically active agent from a microsphere may be increased or decreased depending on the type and/or ratio of PLGA copolymer(s) that forms the microsphere. According to a the present invention, blending RG (75/25) with RG502 can slow the rate of release. For example, compositions including 25-80% RG502 and 20-75% RG(75/25) have slower release rates than either RG520 or RG(75/25) alone (see FIGS. 5 and 6, e.g., FIG. 5 (panel A) and FIG. 6 (panel B)).

The microspheres of the present invention, which have diameters of about 20 nanometers to 300 micrometers and include a biologically active agent, are generated by a double emulsion method. This method provides microspheres with better quality control and a longer half-life. It will be appreciated that any biologically active agent may be included in the inventive microspheres, some of which are provided herein. However, particularly preferred biologically active agents include calcitonin, estrogen, progesterone, and combinations of estrogen and progesterone. Calcitonin is used herein merely for the purpose of exemplification. Any biologically active agent may be substituted for calcitonin. Those skilled in the art would know how to test the dosages of biologically active agent to include in the inventive composition, as such techniques are standard in the art.

Microspheres of about 20 nanometers to 300 micrometers, which contain calcitonin, may be generated by a) contacting a solution of calcitonin with a solution of PLGA copolymer to form a calcitonin:copolymer mixture, wherein the solution of PLGA copolymer includes a blend of copolymers of 25-80% RG502 and 20-75% RG(75/25); b) emulsifying the mixture by sonication to generate a first emulsified solution; c) emulsifying the first emulsified solution by homogenization to form a double emulsified solution; and d) removing the microspheres from the double emulsified solution. As described herein, preferred blends of PLGA copolymers include blends of RG502 and RG(75/25) at ratios of 1:4, 1:1, and 3:1 RG(75/25) to RG502 and the like. Other encapsulation/emulsification techniques familiar to those skilled in the art, including sonication, single emulsification, spray drying, coacervation, and phase inversion, may also be used to generate and isolate microcapsules for use with the invention.

Biologically Active Agents

The present invention provides microsphere delivery systems for biologically active agents. Particularly preferred microsphere delivery systems of the present invention include microsphere delivery systems for proteins (e.g., enzymes), peptides, genetic products (for example, polynucleotides, oligonucleotides and other nucleic acids such as locked nucleic acids (LNAs) and peptide nucleic acids (PNAs) see, e.g., Demidov (supra) and natural or synthetic chemical compounds. For example, the microsphere delivery systems of the invention could deliver calcitonin for the treatment of osteoporosis. Osteoporosis is a disease that affects many older people worldwide, particularly older women. Treatment of osteoporosis in the United States costs an estimated $10 billion annually (Consensus development conference: diagnosis, prophylaxis, and treatment of osteoporosis, American J. Med. (1993) 94:646-650). Other preferred microsphere delivery systems augment hormonal replacement therapy in post-menopausal women. In related embodiments, the present invention provides microsphere delivery systems for the treatment of menopausal symptoms that contain estrogen, progesterone, or a combination of estrogen and progesterone. Other preferred biologically active agent are provided in Table I.

TABLE 1
Bioengineered Pharmaceuticals

Erythropoietins
Interferons
Recombinant Vaccines
Colony Stimulating Factors
Human Insulin
Bioengineered Thrombolytic
Human Growth Hormone
Recombinant Enzymes
Recombinant Blood Factors
Protease Inhibitors
Interleukins
Recombinant Growth Factors

Other biologically active agents that can be incorporated into the microsphere delivery systems of the invention include gastrointestinal therapeutic agents such as aluminum hydroxide, calcium carbonate, magnesium carbonate, sodium carbonate and the like; non-steroidal anti-fertility agents; parasympathomimetic agents; psychotherapeutic agents; major tranquilizers such as chlorpromaxine HCl, clozapine, mesoridazine, metiapine, reserpine, thioridazine and the like; minor tranquilizers such as chlordiazenpoide, diazenpam meprobamate, temazepam and the like; rhinological decongestants; sedative-hynotics such as dodeine, phenobarbital, sodium pentobarbital, sodium secobarbital and the like; steroids such as testosterone and testosterone propionate; sulfonamides; sympathoomimetic agents; vaccines; vitamins and nutrients such as the essential amino acids; essential fats and the like; antimalarials such as 4-aminoquinolines, 8-aminoquinolines, pyrimethaamine and the like, anti-migraine agents such as mazindol, phentermine and the like; anti-Parkinson agents such as L-dopa; anti-spasmodics such as atropine, methscopolamine bromide and the like; antispasmodics and anticholinergic agents such as bile therapy, digestants, enzymes and the like; antitussives such as dextromethorphan, noscapine and the like; broncholdilators; cardiovascular agents such as anti-hypertensive compounds, Rauwolfia alkaloids, coronary vasodilators, nitroglycerin, organic nitrates, pentaerythritotetranitrate and the like; electrolyte replacements such as potassium chloride; ergoalkaloids such as ergotamine with and without caffeine, hydrogenated ergot alkaloids, dihydroergoenstine methanesulfate, dihydroergocorine methanesulfonate, dihydroergokroypine methanesulfate and combinations thereof; alkaloids such as atropine sulfate, Belladotna, hyoscine hydrobromide and the like; analgetics, narcotics such as codeine, dihydrocodienone, meperidine, morphine and the like; non-narcotics such as salicylates, aspirin, acetaminophen, d-propoxyphene and the like; antibiotics, such as cephalosporins, choranphenical, gentimicin, Kanamycin A, Kanamycin B, the penicillins, ampicillin, streptomycin A, antimycin A, chloropamtheniol, metromidazole, oxytetracycline penicillin G, the tetracylines and the like; anti-cancer agents; anticonvulsants such as mephenytoin, phenobarbital, trimethadione; anti-emetics such as thiethylperazine; antihistamines such as cholorophinazine, dimenhydrinate, diphenhycramine, perphenazine, tripelennamine and the like; anti-inflammatory agents such as hormonal agents, hydrocortisone, prednisolone, prednisone, non-hormonal agents, allopurinol, aspirin, indomethacin, phenylbutazone and the like; prostaglandins; cytotoxic drugs such as thiotepa; chlorambucil, cyclophosphamide, melphalan, nitrogen mustard, methotrexate and the like; antigents of such microorganisms as Nisseria gonnorhea, mycobacterium tuberculosis, Herpese virus (humonis types 1 and 2), Candida albicans, Candicla tropicalis, Trichomonas vaginalis, Haemophilus vaginalis, Group B Streptococcus, E. Coli, Microplasma hominis, Hemophilus ducreyi, Granuloma inguinale, Lymphopathia venereum, Treponema pallidum, Brucella abortus, Brucella melitensis, Brucella suis, Brucella canis, Campylobacter fetus, Campylobacter fetus intestinalis, Leptospira pomona, Listeria monocytogenes, Brucella ovis, Equine herpes virus 1, Equine arteritis virus, IBR-IBP virus, BVD-MB virus, Chiamydia psittaci, Trichomonasfoetus, Toxoplasma gondii, Escerichia coli, Actinobacillus equuli, Salmonella abortus ovis, Salmonella aborus equi, Pseudomonas aeruginosa, Corynebacterium equi, Corynebacterium pyogenes, Actinobaccilus seminis, Mycoplasma bovigenitalium, Asperigillus fumigastus, Absidia ramosa, Trypanosoma equiperdun, Babesia caballi, Clostridium tetani and the like; antibodies that counteract the above microorganisms; and enzymes such as ribonucleae, neuramininase, trypsin, glycogen phosphorylase, sperm lactic dehydrogenase, sperm hyaluronidase, adenosinetriphosphatease, alkaline phosphatase, alkaline phosphatase esterase, amino peptidase, trypsin, chymotrypsin, amylase, muramidase, acrosomal proteinase, diesterase, glutarnic acid dehydrogenase, succinic acid dehydrogenase, beta-ol-dehydrogenase, and DPN-di-aprorasse.

Pharmaceutical Compositions

The present invention provides pharmaceutical compositions containing the inventive microspheres. Particularly preferred pharmaceutical compositions include pharmaceutical composition for treating osteoporosis, including an effective amount of calcitonin-containing microspheres. Other preferred pharmaceutical compositions include pharmaceutical compositions for treating or decreasing symptoms of menopause or post-menopause, which include an effective amount of estrogen-containing microspheres. Pharmaceutical compositions for treating menopause can further include microspheres, wherein the biologically active agent is progesterone or a combination of estrogen and progesterone.

The present invention further provides pharmaceutical compositions that include the inventive microspheres, as described in detail above, and a pharmaceutically acceptable carrier. It will be appreciated that the inventive pharmaceutical compositions encompasses the use of any microsphere that is capable of stimulating a biological response, particularly those that increase bone density or ease the symptoms of menopause. It will also be appreciated that certain microspheres of the present invention can exist in free form for treatment.

As described above, the pharmaceutical compositions of the present invention additionally include a pharmaceutically acceptable carrier, which, as used herein, include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington 's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the microspheres of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil, glycols, such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can also be present in the composition, according to the judgment of the formulator.

Administration of Pharmaceutical Compositions

The compositions of the present invention may be employed to induce, stimulate, support, or enhance a biological response in any animal. Preferably, the animal is a domesticated mammal (e.g., a dog, cat, horse, sheep, pig, goat, cow, bird, or lower mammal etc); more preferably, the mammal is a human. In a related embodiment, the compositions may be used to treat or prevent osteoporosis. Individuals to whom the inventive compositions may be administered include any animal or human having been diagnosed as having osteoporosis. For example, any individual who suffers from osteoporosis or who is at risk of developing osteoporosis may be treated. It will be appreciated that an individual can be considered at risk for developing osteoporosis without having been diagnosed with any symptoms of osteoporosis. For example, the individual may have a particular genetic marker identified as being associated with increased risk for developing osteoporosis. Similarly, if members of an individual's family have been diagnosed with osteoporosis, the individual may be considered to be at risk for developing osteoporosis. In addition, an individual who has been identified to have decreased bone density may be an individual at risk for developing osteoporosis. In another related embodiment, the compositions may be used to treat or prevent the symptoms of menopause. In yet other embodiments, pharmaceutical compositions described herein may also be used to treat cancer.

The compositions of the present invention may be formulated for delivery by any route. Preferably, the compositions are formulated for injection. Alternatively, the compositions are formulated for ingestion or inhalation. The compositions are administered in such amounts and for such time as necessary to achieve the desired result. As described above, in certain embodiments of the present invention a “therapeutically effective amount” or an “effective amount” of an inventive pharmaceutical composition is that amount effective for attenuating, stimulating, supporting, or enhancing a biological response in any animal. The inventive mixtures and compositions, according to the method of the present invention, may be administered using any amount and any route of administration effective for stimulating, supporting, or enhancing a biological response in any animal. Thus, the “amount effective to attenuate, stimulate, support, or enhance a biological response in any animal,” as used herein, refers to a nontoxic but sufficient amount of the inventive microspheres or pharmaceutical composition containing microspheres to stimulate, support, or enhance a biological response in any animal. As but one example, the “therapeutically effective amount” can be an amount to treat or prevent osteoporosis. Alternatively, the inventive microspheres can be used to attenuate the symptoms of menopause.

The exact amount required will vary from subject to subject, depending on the species, age, and general condition of the subject, the stage of the disease, the particular microsphere, its mode of administration and the like. The microspheres of the invention are preferably formulated in dosage unit form for ease of administration and uniformity of dosage. The expression “dosage unit form” as used herein refers to a physically discrete unit of microspheres appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific biologically active agent employed; the specific composition employed; the age, body weight, general health, sex and diet of the patient; the time of administration, route of administration, and rate of excretion of the specific biologically active agent employed; the duration of the treatment; drugs used in combination or coincidental with the specific compound employed; and like factors well known in the medical arts.

In particularly preferred embodiments, the microspheres are formulated for injection. Most preferably, the microspheres are implanted into a specific in vivo location by injection to that location. Injection of the pharmaceutical compositions of the invention is to a site in the body that will stimulate a biological response in an individual. Alternatively, injection of the pharmaceutical compositions of the invention to a site in the body where it is desired that a biological response be stimulated, supported, or enhanced may be particularly effective. Injection of the inventive pharmaceutical composition directly into or around a bone, for example, may result in a biological response at the site of osteoporosis, e.g., to increase bone density.

For example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

In other embodiments, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the location or severity of the condition being treated. In certain embodiments, the compounds of the invention may be administered orally or parenterally at dosage levels of biologically active agent of about 0.1 mg/kg to about 50 mg/kg and preferably from about 2 mg/kg to about 25 mg/kg, of patient body weight per day, one or more times a day, to obtain the desired therapeutic effect. Of course a greater quantity of biologically active agent can be included if the biologically active agent is released over time, so no toxic side effects are induced in the first burst.

Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Although not required, the active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-cancer agent), or they may achieve different effects.

For example, the microspheres may be co-administered with other anti-osteoporosis agents or other agents for the treatment of menopausal symptoms. In addition, the micropheres of the invention may be co-administered with other compounds for treatment of other unrelated conditions.

Other biologically active agents may be administered prior to, concurrently with, or after administration of an inventive pharmaceutical composition. For example, the inventive microspheres may be administered alone or in combination with one or more additional factors, such as calcium pills, hormones, etc., in order to enhance the effectiveness of the overall treatment.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use, or sale for human administration.

Applications

The microspheres of the present invention have application in the field of drug delivery, particularly protein and peptide drugs delivery. In preferred embodiments, the present invention provides microspheres for delivery of protein and peptide pharmaceuticals in vivo. In particularly preferred embodiments, the present invention provides microspheres that release calcitonin for the treatment of osteoporosis. Calcitonin delivery can also be used to augment hormone replacement therapy in menopausal or post-menopausal women. The present invention achieves release of the compound without chemical modification or addition of excipients in the pharmaceutical composition. The physical blending of PLGA copolymers was found to be an effective means to control or alter protein release characteristics such as the initial burst effect, duration of release, timing of release, and amount of release of the biologically active agent from the microsphere.

Those skilled in the art will appreciate that the effectiveness of the microspheres of the present invention in eliciting certain biological responses in vivo can be tested in any relevant in vitro cell culture assay or in vivo in appropriate animal models. For example, the effectiveness of calcitonin delivery may be assessed using a mouse model for osteoporosis (see, e.g., Baltzer et al. Gene Ther (2001) 8(23):1770-1776 or Bolon et al., Mol. Ther. (2001) 3(2):197-205). Use of the inventive microspheres for hormone replacement therapy can also be measured in an animal model (see Raafat et al. J. Cell Physiol. (2001) 187(1):81-89 or Raafat et al. Am. J. Obstet. Gynecol (2001) 184(3):340-349)

Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance, which can be adapted to the practice of this invention in its various embodiments and the equivalents thereof.

### EXAMPLES

### Example 1

### Production of Microspheres

In order to understand the releasing behavior of different protein encapsulated microspheres made of different PLGA compositions, bovine serum albumin (BSA) was selected as a model for larger protein macromolecules and calcitonin was selected as a model for smaller macromolecules. BSA has a molecular weight of 66 KDal and is relatively stable in processing and storage (Hongkee Sah, Protein behavior at the water/methylene chloride interface. Science (1999) 88(12):1320-1325). Calcitonin is a 4.5 KDal, 32 amino acid protein, which is a potential drug for therapy of osteoporosis. The sequence of salmon calcitonin used in these examples is shown below.

TABLE 2
Salmon Calcitonin Amino Acid Sequence

H-Cys-Ser-Asn-Leu-Ser-Thr-Cys-Val-Leu-Gly-
Lys-Leu-Ser-Gln-Glu-Leu-His-Lys-Leu-Gln-
Thr-Tyr-Pro-Arg-Thr-Asn-Thr-Gly-Ser-Gly-
Thr-Pro-NH₂

Materials

Poly(DL-lactide-co-glycolide) copolymers including RG502, RG503, RG503H, and RG(75/25) were purchased from Boehringer Ingelheim. Polyvinylalcohol (MW 9000-10000, 80% hydrolyzed) was purchased from Aldrich Chemical. BSA and calcitonin were purchased from Sigma. All other solvents including methylene chloride and acetone are analytical grade.

Protein solutions were prepared of calcitonin and BSA. A calcitonin solution was prepared by dissolving five mg of calcitonin in 0.5 mL 30% acetic acid. A BSA solution was prepared by dissolving 120 mg of BSA in 600 mL water. A PLGA solution was prepared by dissolving 80 mg of PLGA in 0.5 mL methylene chloride for forming microspheres with calcitonin; or dissolving 200 mg of PLGA in 1 mL methylene chloride for forming microspheres with BSA. A solution of PVA was prepared by dissolving 50 g PVA in 1 L distilled water as a stock solution. When it was used in a second emulsion, it was diluted five times. All solutions were kept in the refrigerator.

Preparation of Microspheres

Forty mL of the calcitonin solution was added to 0.5 ml PLGA solution; or 100 mL BSA was added to 1 mL PLGA solution. The combined solution was emulsified by sonicate for 5-6 pulses. The first emulsified solution was poured into 100 ml 1% PVA solution and homogenized by a homogenizer for one minute. The homogenization was at 5100 rpm for BSA and 3100 rpm for calcitonin. The double emulsified solution was let stand to evaporate the methylene chloride and harden the microspheres under stirring for 2-3 hours. Microspheres were collected by centrifuging the microsphere solution at 1500 rpm for six minutes. The microspheres were washed with 30 mL distilled water twice. Finally, the microspheres were lyophilized for more than four days. The particle size of the microspheres was analyzed by Couter^{R} Multisizer AccuSomp^{R} 1.15.

In Vitro Release Assay

To determine release of calcitonin or BSA protein in vitro, 10 mg of microspheres were suspended in 1.0 mL of isotonic PBS pH 7.4. The samples were kept shaking in vials at 37° C. The liquid samples were periodically collected, centrifuged, and the protein concentration of the supernatants determined by BCA kit (Pierce Pharmaceuticals, Rockford Illinois). For continuous release assays, another 1 mL of isotonic PBS pH 7.4 was added.

Results

The BSA encapsulated microspheres had a mean particle size of between 30 and 40 micrometers (see Table 2).

TABLE 2
Mean Size of BSA Microspheres

| Microspheres | Mean Size(micrometers |
|---|---|
| RG502 | ˜29 |
| RG503 | ˜38 |
| RG503H | ˜37 |
| RG(75/25) | ˜39 |
| 75% RG(75/25) and 25% RG502 | ˜39 |
| 50% RG(75/25) and 50% RG502 | ˜37 |

These results indicate that the double emulsion process is highly reproducible.

### OTHER EMBODIMENTS

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims. For example, the following paragraphs represent other possible embodiments of the invention.

1. A composition of microspheres comprising a blend of polyhydroxy acids and a biologically active agent.

2. The composition of claim 1, wherein the blend of polyhydroxy acids comprises a blend of polylactic acid and polyglycolic acid and copolymers thereof.

3. The composition of claim 2, wherein the blend of polylactic acid and polyglycolic acids comprises poly(D,L-lactide-co-glycolide) and poly(D,L-lactide-co-glycolide).

4. The composition of claim 1, wherein the blend comprises poly-ε-lactone and copolymers thereof.

5. The composition of claim 3, wherein the blend is a 1:4 ratio of poly(D,L-lactide-co-glycolide), which has a 75%:25% ratio of racemic lactide DL to glycolide and a molecular weight of 2 (RG(75/25)), to poly(D,L-lactide-co-glycolide), which has a 50%:50% ratio of racemic lactide DL to glycolide and a molecular weight of 20 Kdal (RG502).

6. The composition of claim 3, wherein the blend is a 1:1 ratio of RG(75/25) to RG502.

7. The composition of claim 3, wherein the blend is a 3:1 ratio of RG(75/25) to RG502.

8. The composition of any one of claims 1-7, wherein the biologically active agent comprises calcitonin.

9. The composition of any one of claims 1-7, wherein the biologically active agent comprises estrogen.

10. The composition of any one of claims 1-7, wherein the biologically active agent comprises progesterone.

11. The composition of any one of claims 1-7, wherein the biologically active agent comprises a combination of estrogen and progesterone.

12. A pharmaceutical composition for treating osteoporosis comprising an effective amount of calcitonin-containing microspheres, wherein the microspheres comprise a blend of poly(DL-lactide-co-glycolide) (PLGA) copolymers.

13. The pharmaceutical composition of claim 12, wherein the blend of PLGA copolymers comprises 25-80% RG502 and 20-75% RG(75/25).

14. The pharmaceutical composition of claim 12, wherein the copolymer comprises a 1:4 ratio of RG(75/25) to RG502.

15. The pharmaceutical composition of claim 12, wherein the copolymer comprises a 1:1 ratio of RG(75/25) to RG502.

16. The pharmaceutical composition of claim 12, wherein the copolymer comprises a 3:1 ratio of RG(75/25) to RG502.

17. The composition of claim 12, further comprising a hydrophilic acid end group.

18. The composition of claim 12, further comprising a lipophilic ester end group.

19. A pharmaceutical composition for treating symptoms of menopause comprising an effective amount of estrogen containing microspheres, wherein the microspheres comprise a blend of poly(DL-lactide-co-glycolide) (PLGA) copolymers.

20. The pharmaceutical composition of claim 19, wherein the blend of PLGA copolymers comprises 25-80% RG502 and 20-75% RG(75/25).

21. The pharmaceutical composition of claim 20, wherein the copolymer comprises a 1:4 ratio of RG(75/25) to RG502.

22. The pharmaceutical composition of claim 19, wherein the copolymer comprises a 1:1 ratio of RG(75/25) to RG502.

23. The pharmaceutical composition of claim 19, wherein the copolymer comprises a 3:1 ratio of RG(75/25) to RG502.

24. The composition of claim 19, further comprising a hydrophilic acid end group.

25. The composition of claim 19, further comprising a lipophilic ester end group.

26. A pharmaceutical composition for treating symptoms of menopause comprising an effective amount of a combination of estrogen and progesterone containing microspheres, wherein the microspheres comprise a blend of poly(DL-lactide-co-glycolide) (PLGA) copolymers.

27. The pharmaceutical composition of claim 26, wherein the blend of PLGA copolymers comprises 25-80% RG502 and 20-75% RG(75/25).

28. The pharmaceutical composition of claim 27, wherein the copolymer comprises a 1:4 ratio of RG(75/25) to RG502.

29. The pharmaceutical composition of claim 26, wherein the copolymer comprises a 1:1 ratio of RG(75/25) to RG502.

30. The pharmaceutical composition of claim 26, wherein the copolymer comprises a 3:1 ratio of RG(75/25) to RG502.

31. The composition of claim 26, further comprising a hydrophilic acid end group.

32. The composition of claim 26, further comprising a lipophilic ester end group.

33. A method of treating osteoporosis comprising administering a composition of microspheres comprising a blend of poly(DL-lactide-co-glycolide) copolymers and calcitonin.

34. A method of decreasing symptoms associated with menopause comprising administering a composition of microspheres comprising a blend of poly(DL-lactide-co-glycolide) copolymers and estrogen.

35. A method of decreasing symptoms associated with menopause comprising administering a composition of microspheres comprising a blend of poly(DL-lactide-co-glycolide) copolymers and a combination of estrogen and progesterone.

36. The method of claim 33, 34, or 35 wherein the blend of poly(DL-lactide-co-glycolide) copolymers comprises 25-80% RG502 and 20-75% RG(75/25).

37. The method of claim 33, 34, or 35, wherein the blend of poly(DL-lactide-co-glycolide) copolymers comprises a 1:4 ratio of RG(75/25) to RG502.

38. The method of claim 33, 34, or 35, wherein the blend of poly(DL-lactide-co-glycolide) copolymers comprises a 1:1 ratio of RG(75/25) to RG502.

39. The method of claim 33, 34, or 35, wherein the blend of poly(DL-lactide-co-glycolide) copolymers comprises a 3:1 ratio of RG(75/25) and RG502.

40. A method of generating controlled release microspheres containing biologically active agents:

blending poly(lactic-co-glycolytic acid) polymers of varying molecular weight to form a copolymer, wherein the diameter of the polymer varies from 20 nanometers to 300 micrometers.

41. The method of claim 40, wherein the lactic acid-glycolic acid ratio is selected from the group of ratios consisting of 1:2, 1:3, 1:4, and 1:5.

42. A method of controlling the rate of release of a biologically active agent from a microsphere comprising varying the type of poly(DL-lactide-co-glycolide) copolymer in a microsphere.

43. A method of controlling the cumulative release of a biologically active agent from a microsphere comprising varying the type of poly(DL-lactide-co-glycolide) copolymer in a microsphere.

44. A method of controlling the duration of release of a biologically active agent from a microsphere comprising varying the type of poly(DL-lactide-co-glycolide) copolymer in a microsphere.

45. A method of controlling the burst effect of a biologically active agent from a microsphere comprising varying the type of poly(DL-lactide-co-glycolide) copolymer in a microsphere, wherein the burst effect comprises a first and a second pulse.

46. The method of claim 45, wherein the type of poly(DL-lactide-co-glycolide) copolymer controls the timing of the first pulse.

47. The method of claim 45, wherein the type of poly(DL-lactide-co-glycolide) copolymer controls the amount released by the first pulse

48. The method of claim 45, wherein the type of poly(DL-lactide-co-glycolide) copolymer controls the duration of the first pulse.

49. The method of claim 45, wherein the type of poly(DL-lactide-co-glycolide) copolymer controls the timing of the second pulse.

50. The method of claim 45, wherein the type of poly(DL-lactide-co-glycolide) copolymer controls the amount of biologically active agent released by the second pulse.

51. The method of claim 45, wherein the type of poly(DL-lactide-co-glycolide) copolymer controls the duration of the second pulse.

52. The method of claim 42, wherein the type of poly(DL-lactide-co-glycolide) copolymer is a blend of PLGA copolymers.

53. The method of claim 52, wherein the type of type of poly(DL-lactide-co-glycolide) copolymer is a blend of RG(75/25) and RG502.

54. The method of claim 53, wherein the blend of PLGA copolymers comprises a 1:4 ratio of RG(75/25) to RG502.

55. The method of claim 53, wherein the blend of PLGA copolymers comprises a 1:1 ratio of RG(75/25) to RG502.

56. The method of claim 53, wherein the blend of PLGA copolymers comprises a 3:1 ratio of RG(75/25) to RG502.

57. The method of claim 53, wherein the blend of PLGA copolymers comprises 25-80% RG502 and 20-75% RG(75/25).

58. The method of claim 41, 42, 43, 44, or 45, wherein the biologically active agent is selected from the group consisting of calcitonin, estrogen, progesterone, and a combination of estrogen and progesterone.

59. A method of controlling the rate of release of a biologically active agent from a microsphere comprising varying the ratio of a first to a second poly(DL-lactide-co-glycolide) (PLGA) copolymer in a microsphere, wherein the first and second PLGA copolymers have different ratios of lactide and glycolide.

60. A method of controlling the cumulative release of a biologically active agent from a microsphere comprising varying the ratio of a first to a second poly(DL-lactide-co-glycolide) copolymer in a microsphere.

61. A method of controlling the duration of release of a biologically active agent from a microsphere comprising varying the ratio of a first to a second poly(DL-lactide-co-glycolide) copolymer in a microsphere.

62. A method of controlling the burst effect of a biologically active agent from a microsphere comprising varying the ratio of a first to a second poly(DL-lactide-co-glycolide) copolymer in a microsphere, wherein the burst effect comprises a first pulse and a second pulse.

63. The method of claim 62, wherein the ratio of the first and second poly(DL-lactide-co-glycolide) copolymer controls the timing of the first pulse.

64. The method of claim 62, wherein the ratio of the first and second poly(DL-lactide-co-glycolide) copolymer controls the amount of biologically active agent released by the first pulse.

65. The method of claim 62, wherein the ratio of the first and second poly(DL-lactide-co-glycolide) copolymer controls the duration of the first pulse.

66. The method of claim 62, wherein the ratio of the first and second poly(DL-lactide-co-glycolide) copolymer controls the timing of the second pulse.

67. The method of claim 62, wherein the ratio of the first and second poly(DL-lactide-co-glycolide) copolymer controls the amount of biologically active agent released by the second pulse.

68. The method of claim 62, wherein the ratio of the first and second poly(DL-lactide-co-glycolide) copolymer controls the duration of the second pulse.

69. The method of claim 59, wherein the ratio of the first to the second poly(DL-lactide-co-glycolide) copolymer is a blend of PLGA copolymers.

70. The method of claim 69, wherein the blend of poly(DL-lactide-co-glycolide) copolymer is a blend of RG(75/25) and RG502.

71. The method of claim 69, wherein the blend of PLGA copolymers comprises a 1:4 ratio of RG(75/25) to RG502.

72. The method of claim 70, wherein the blend of PLGA copolymers comprises a 1:1 ratio of RG(75/25) to RG502.

73. The method of claim 70, wherein the blend of PLGA copolymers comprises a 3:1 ratio of RG(75/25) to RG502.

74. The method of claim 70, wherein the blend of PLGA copolymers comprises 25-80% RG502 and 20-75% RG(75/25).

75. The method of claim 59, 60, 61, or 62, wherein the biologically active agent is selected from the group consisting of calcitonin, estrogen, and a combination of estrogen and progesterone.

76. A method of making microspheres of about 20 nanometers to 300 micrometers in diameter comprising a biologically active agent comprising the steps of:

contacting a solution of a biologically active agent with a solution of poly(DL-lactide-co-glycolide) copolymer to form a calcitonin:copolymer mixture;

emulsifying the mixture by sonication to generate a first emulsified solution;

emulsifying the first emulsified solution by homogenization to form a double emulsified solution; and

removing the microspheres from the double emulsified solution.

77. The method of claim 76, wherein the solution of poly(DL-lactide-co-glycolide) copolymer comprises a blend of copolymers comprising 25-80% RG502 and 20-75% RG(75/25).

78. The method of claim 76, wherein the blend of copolymers comprises a 1:4 ratio of RG(75/25) to RG502.

79. The method of claim 76, wherein the blend of copolymers comprises a 1:1 ratio of RG(75/25) to RG502.

80. The method of claim 76, wherein the blend of copolymers comprises a 3:1 ratio of RG(75/25) to RG502.

81. The method of claim 76, wherein the biologically active agent is estrogen, progesterone, a combination of estrogen and progesterone, or calcitonin.;

82. The composition of claim 76, further comprising a hydrophilic acid end group.

83. The composition of claim 76, further comprising a lipophilic ester end group.

1. A composition of microspheres comprising a blend of biodegradable polymers and a biologically active agent, wherein the composition of the blend modulates the release kinetics of the microspheres.

2. The composition of claim 1, wherein the release kinetics comprises rate of release of the biologically active agent from the microsphere.

3. The composition of claim 1, wherein the release kinetics comprises cumulative release of the biologically active agent from the microsphere.

4. The composition of claim 1, wherein the release kinetics comprises duration of release of the biologically active agent from the microsphere.

5. The composition of claim 1, wherein the release kinetics comprises burst effect of the biologically active agent from a microsphere.

6. The composition of claim 1, wherein the release kinetics comprises timing of release of the biologically active agent by a first pulse.

7. The composition of claim 1, wherein the release kinetics comprises amount of the biologically active agent released by the first pulse.

8. The composition of claim 1, wherein the release kinetics comprises duration of release of the biologically active agent released by a first pulse.

9. The composition of claim 1, wherein the release kinetics comprises timing of release of the biologically active agent release by a second pulse.

10. The composition of claim 1, wherein the release kinetics comprises amount of the biologically active agent released by a second pulse.

11. The composition of claim 1, wherein the release kinetics comprises duration of release of the biologically active agent by a second pulse.

12. The composition of claim 1, further comprising a blend of polyhydroxy acids and a biologically active agent.

13. The composition of claim 12, wherein the blend of polyhydroxy acids comprises a blend of polylactic acid and polyglycolic acid and copolymers thereof.

14. The composition of claim 13, wherein the blend of polylactic acid and polyglycolic acids comprises poly(D,L-lactide-co-glycolide).

15. The composition of claim 12, wherein the blend comprises poly-ε-lactone and copolymers thereof.

16. The composition of claim 14, wherein the blend is a 1:4 ratio of poly(D,L-lactide-co-glycolide), which has a 75%:25% ratio of racemic lactide DL to glycolide and a molecular weight of 20 Kdal (RG(75/25)), to poly(D,L-lactide-co-glycolide), which has a 50%:50% ratio of racemic lactide DL to glycolide and a molecular weight of 20 Kdal (RG502).

17. The composition of claim 14, wherein the blend is a 1:1 ratio of RG(75/25) to RG502.

18. The composition of claim 14, wherein the blend is a 3:1 ratio of RG(75/25) to RG502.

19. The composition of any one of claims 12-18, wherein the biologically active agent comprises calcitonin.

20. The composition of any one of claims 12-18, wherein the biologically active agent comprises estrogen.

21. The composition of any one of claims 12-18, wherein the biologically active agent comprises progesterone.

22. The composition of any one of claims 12-18, wherein the biologically active agent comprises a combination of estrogen and progesterone.

23. A method of controlling the rate of release of a biologically active agent from a microsphere comprising varying the ratio of a first to a second poly(DL-lactide-co-glycolide) (PLGA) copolymer in a microsphere, wherein the first and second PLGA copolymers have different ratios of lactide and glycolide.

24. A method of controlling the cumulative release of a biologically active agent from a microsphere comprising varying the ratio of a first to a second poly(DL-lactide-co-glycolide) copolymer in a microsphere.

25. A method of controlling the burst effect of a biologically active agent from a microsphere comprising varying the ratio of a first to a second poly(DL-lactide-co-glycolide) copolymer in a microsphere, wherein the burst effect comprises a first pulse and a second pulse.

26. The method of claim 25, wherein the ratio of the first and second poly(DL-lactide-co-glycolide) copolymer controls the timing of the first pulse.

27. The method of claim 25, wherein the ratio of the first and second poly(DL-lactide-co-glycolide) copolymer controls the amount of biologically active agent released by the first pulse.

28. The method of claim 25, wherein the ratio of the first and second poly(DL-lactide-co-glycolide) copolymer controls the duration of the first pulse.

29. The method of claim 25, wherein the ratio of the first and second poly(DL-lactide-co-glycolide) copolymer controls the timing of the second pulse.

30. The method of claim 25, wherein the ratio of the first and second poly(DL-lactide-co-glycolide) copolymer controls the amount of biologically active agent released by the second pulse.

31. The method of claim 25, wherein the ratio of the first and second poly(DL-lactide-co-glycolide) copolymer controls the duration of the second pulse.

32. The method of claim 25, wherein the ratio of the first to the second poly(DL-lactide-co-glycolide) copolymer is a blend of PLGA copolymers.
